(19) United States
(12) Patent Application Publication
(10) Pub. No.: US 2019/0236977 A1
(43) Pub. Date: Aug. 1, 2019

(54) BEHAVIOR MODIFICATION METHOD

(71) Applicant: Pavel Pavlovich Horoshutin, Irkutsk (RU)

(72) Inventor: Pavel Pavlovich Horoshutin, Irkutsk (RU)

(21) Appl. No.: 16/378,587

(22) Filed: Apr. 9, 2019

Related U.S. Application Data
(63) Continuation-in-part of application No. 16/255,176, filed on Jan. 23, 2019.
Application No. 16/255,176 is a continuation-in-part of application No. PCT/RU2018/000295, filed on May 7, 2018.
Application No. 16/255,176 is a continuation-in-part of application No. PCT/RU2017/000008, filed on Jan. 11, 2017.

(30) Foreign Application Priority Data
Jul. 25, 2016 (RU) 2016130360
Jan. 24, 2018 (RU) 2018102742

(51) Int. Cl. G09B 19/00; A61B 5/00; G09B 5/06; A61B 5/16

(52) U.S. Cl. CPC G09B 19/0092; A61B 5/0022; A61B 5/165; G09B 19/00; A61B 5/167; G09B 5/065

(57) ABSTRACT

Systems, methods, and kits for behavior modification are described herein. A behavior modification method may commence with performing psychodiagnostics of a patient to provide psychodiagnostics data associated with the patient. The method may further include determining dominance hierarchy of sensory channels of the patient and an attitude pattern of undesirable behavior of the patient. The method may further include developing an attitude pattern of desirable behavior for the patient. The method may continue with selecting at least one unconditioned stimulus and at least one conditioned stimulus for the patient based on the dominance hierarchy of sensory channels. The method may further include selecting a kit for the patient to undergo a behavior modification training. The method may further include performing the behavior modification of the patient by conducting the behavior modification training by the patient using one or more of the plurality of items of the kit.

### CROSS-REFERENCE TO RELATED APPLICATIONS

This application is a Continuation-in-part of U.S. patent application Ser. No. 16/255,176, entitled “METHOD OF PROVIDING REMOTE PSYCHOLOGICAL AID,” filed on Jan. 23, 2019, which is a Continuation-in-part of PCT application No. PCT/RU2017/000008, entitled “METHOD OF PROVIDING REMOTE PSYCHOLOGICAL AID”, filed on Jan. 11, 2017, which claims priority to Russian Application No. 201613036, entitled “METHOD OF PROVIDING REMOTE PSYCHOLOGICAL AID”, filed on Jul. 25, 2016, and is a Continuation-in-part of PCT application No. PCT/RU2018/000295, entitled “METHOD OF PROVIDING REMOTE PSYCHOLOGICAL AID”, filed on May 7, 2018, which claims priority to Russian Application No. 2018102742, entitled “METHOD OF PROVIDING REMOTE PSYCHOLOGICAL AID”, filed on Jan. 24, 2018. The aforementioned applications are incorporated herein by reference in their entireties for all purposes.

### TECHNICAL FIELD

The present disclosure relates generally to cognitive behavioral psychology and behavioral psychology and psychotherapy, and specifically to methods for behavior modification to solve problems related to problematic social behavior, dependent behavior, and behavior provoking psychosomatic diseases.

### BACKGROUND

Classical conditioning is a learning process in which a neutral stimulus is repeatedly paired with an unconditioned stimulus resulting in formation of an association between the neutral stimulus and the unconditioned stimulus. Upon creation of the association, the previously neutral stimulus becomes a conditioned stimulus. The conditioned stimulus is also referred to a conditioned reflex, and the unconditioned stimulus is also referred to an unconditioned reflex. The conditioned reflex and unconditioned reflex were discovered and described by physiologist I. P. Pavlov.

The unconditioned reflex is a congenital inherited stereotypical body reaction to the influence of the internal or environmental environment. The unconditioned reflex persists in a person throughout a person's life. Reflex arcs pass through the brain and spinal cord. The conditioned reflex is an acquired reflex of a particular person. The conditioned reflex is created during the life of the person and is not fixed genetically. It may be created under certain conditions and disappears in absence of these conditions. The conditioned reflex is formed based on unconditioned reflexes with the participation of the higher brain regions and, specifically, the cerebral cortex. The conditioned reflex reactions depend on past experience of a person and specific conditions in which the conditioned reflex is formed. A first order conditioned reflex is a steady conditioned reflex that can be used to condition a new conditioned reflex.

Currently, many behavior modification methods exist. One example behavior modification method is based on operant conditioning. The operant conditioning method may be illustrated by classical modification behavior developed by B. F. Skinner. Specifically, this method may be described by the formula: previous behavior—modification process—new behavior. In case of the operant conditioning method, the previous behavior is searching for food, the modification process is based on the unconditioned reflex of searching for food and the conditioned reflex of pushing a lever of a feeder and reinforcing the conditioned reflex by food. The new behavior is opening the feeder using the level.

The disadvantages of operative conditioning methods include the high labor intensity of these methods for a therapist. These disadvantages result from the necessity to solve several problems at once. Firstly, it is difficult to select a conditioned stimulus that would solve the tasks of changing the problematic behavior, dependent behavior, and behavior provoking psychosomatic diseases. Secondly, it is difficult to create a behavioral model in which this conditioned stimulus would be steadily present. Thirdly, a person has to be regularly subjected to a therapeutic procedure to timely reinforce the conditioned stimulus. The operative conditioning method can be used only in a limited number of therapeutic cases.

Further behavior modification methods include a classical conditioning method. This method is also described by the formula: previous behavior—modification process—new behavior. Specifically, the previous behavior is a food reaction to food. The modification process includes activating the unconditioned reflex by providing food and selecting a bell as the conditioned reflex. The new behavior is a food reaction to the bell.

The disadvantages of classical conditioning method include high complexity of the process of creating a therapeutic behavioral environment. Furthermore, this method does not have an efficient solution for selecting a conditioned reflex suitable for modifying the old problematic behavior. The classical conditioning method can be used for behavior modification only in a limited number of psychotherapeutic cases.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Provided are methods and systems for behavior modification. In some example embodiments, a behavior modification method may commence with performing psychodiagnostics of a patient to provide psychodiagnostics data associated with the patient. The method may further include determining dominance hierarchy of sensory channels of the patient and an attitude pattern of undesirable behavior of the patient based on the psychodiagnostics data. The method may further include developing an attitude pattern of desirable behavior for the patient based on the attitude pattern of undesirable behavior and the psychodiagnostics data. The method may continue with selecting at least one unconditioned stimulus and at least one conditioned stimulus for the patient based on the dominance hierarchy of sensory channels. The method may further include selecting an item kit for the patient to undergo a behavior modification training. The item kit may include a plurality of items to be physically contacted with by the patient using the sensory channels. The item kit may be selected based on the attitude pattern of desirable behavior, the at least one unconditioned stimulus, and at least one conditioned stimulus. The method may further include performing the behavior modification of the patient. The behavior modification may be performed by conducting the behavior modification training by the patient using one or more of the plurality of items of the item kit.

In some example embodiments, a system for behavior modification may include a data collection unit configured to perform psychodiagnostics of a patient to provide psychodiagnostics data associated with the patient. The system may further include an analyzing unit configured to determine dominance hierarchy of sensory channels of the patient and an attitude pattern of undesirable behavior of the patient based on the psychodiagnostics data. The analyzing unit may be further configured to develop an attitude pattern of desirable behavior for the patient based on the attitude pattern of undesirable behavior and the psychodiagnostics data. The analyzing unit may be further configured to select at least one unconditioned stimulus and at least one conditioned stimulus for the patient based on the dominance hierarchy of sensory channels. The analyzing unit may be further configured to select an item kit for the patient to undergo a behavior modification training, wherein the item kit includes a plurality of items to be physically contacted with by the patient using the sensory channels. The selection of the item kit may be performed based on the attitude pattern of desirable behavior, the at least one unconditioned stimulus, and at least one conditioned stimulus. The system may further include a training unit configured to perform the behavior modification of the patient. The performing of the behavior modification may include conducting the behavior modification training by the patient using one or more of the plurality of items of the item kit.

In some example embodiments, an item kit for behavior modification is provided. The item kit may include a plurality of items and instructions on use of the item kit. The plurality of items may include one or more of the following: a text for audio listening, at least one food additive, at least one aromatic essential oil, at least one mineral, and one or more images with a suggestive formula to develop an attitude pattern of desirable behavior of a patient. Each of the plurality of items may be selected based on psychodiagnostics data of the patient obtained based on psychodiagnostics of the patient. Each of the plurality of items may be selected to act as at least one unconditioned stimulus or at least one conditioned stimulus for the patient using sensory channels of the patient.

Additional objects, advantages, and novel features will be set forth in part in the detailed description section of this disclosure, which follows, and in part will become apparent to those skilled in the art upon examination of this specification and the accompanying drawings or may be learned by production or operation of the example embodiments. The objects and advantages of the concepts may be realized and attained by means of the methodologies, instrumentalities, and combinations particularly pointed out in the appended claims.

### BRIEF DESCRIPTION OF THE DRAWINGS

Embodiments are illustrated by way of example and not limitation in the figures of the accompanying drawings, in which like references indicate similar elements and in which:

FIG. 1 illustrates an environment within which system and methods for behavior modification can be implemented, in accordance with some embodiments.

FIG. 2 is a block diagram showing various modules of a system for behavior modification, in accordance with certain embodiments.

FIG. 3 is a flow chart illustrating a behavior modification method, in accordance with an example embodiment.

FIG. 4 is a schematic diagram illustrating behavior modification of a patient using an item kit, according to an example embodiment.

FIG. 5 shows a computing system that can be used to implement a behavior modification method, according to an example embodiment.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical, and electrical changes can be made without departing from the scope of what is claimed. The following detailed description is, therefore, not to be taken in a limiting sense, and the scope is defined by the appended claims and their equivalents.

The present disclosure provides systems and methods for behavior modification. A method for behavior modification is aimed at solving psychological problems of a person by modifying the person behavior. The method for behavior modification may include performing psychodiagnostics of a patient. Specifically, a request for the behavior modification may be received by a psychological center from the patient. The request may be received via communication means, such as a mail, a phone, a delivery service, the Internet, and so forth. Based on the request received from the patient, one or more tests to perform the psychodiagnostics may be provided to the patient via the communication means. Upon receipt of the tests, the patient may perform the psychodiagnostics by answering questions of the tests. In other words, the testing of the patient may be performed by the patient himself without an in-person meeting with a psychologist of the psychological center. Upon passing the tests, the patient may send results of the tests to the psychological center via the communication means. The received results of the tests may serve as psychodiagnostics data of the patient. Furthermore, results of the tests may be further analyzed to determine psychodiagnostics data of the patient.

Based on the psychodiagnostics data, dominance hierarchy of sensory channel of a sensory system of the patient may be determined. For example, the patient may have one dominant sensory channel, such as a visual sensory channel. Other sensory channels of the patient may be non-dominant. In other words, the patient may react more strongly to environmental information perceived by the patient using the visual sensory channel. For example, the patient may memorize a song lyrics written on a paper sheet and read by the patient, but may memorize less efficiently the song lyrics heard by the patient in a song.

Furthermore, the psychodiagnostics data may be used to determine an attitude pattern of undesirable behavior of the patient. For example, the attitude pattern of undesirable behavior may include overeating and weight gain. In psychology, an attitude includes a set of emotions, behaviors, and beliefs towards a particular person, thing, object, or event. Based on the attitude pattern of undesirable behavior and the psychodiagnostics data of the patient, the psychologist may develop an attitude pattern of desirable behavior for the patient. For example, the attitude pattern of desirable behavior may include an attitude pattern directed on lowering the weight of the patient, such as avoiding eating high-calorie products, eating a predetermined amount of vegetables, and the like.

Based on the dominance hierarchy of sensory channels, an unconditioned stimulus and a conditioned stimulus may be selected for the patient. The unconditioned stimulus may include a food stimulus, and the conditioned stimulus may include a visual stimulus (as a visual sensory channel is a dominant sensory channel of the patient) and, optionally, one or more of an auditory stimulus, a tactile stimulus, an olfactory stimulus, a kinesthetic stimulus, and a gustatory stimulus. The conditioned stimulus may be formed using a second signal system, namely using audio and visual information.

Then, an item kit for the patient to undergo a behavior modification training may be selected. The item kit may include a plurality of items to be contacted with by the patient using the sensory channels. Furthermore, a program of the behavior modification training, such as a list of actions to be performed by the patient using the item kit, may be developed. The item kit and the program may be provided to the patient. Upon providing the item kit and the program to the patient, the behavior modification training may be performed by the patient using the item kit.

The conditioned reflex theory by I. P. Pavlov. The method of behavior modification is based on the conditioned reflex theory by I. P. Pavlov. The conditioned reflex theory relates to studying higher nervous activity. The Pavlov's studies were directed to understanding the work of a human brain and to providing scientific support to phenomena designated as psychic phenomena. The substantiation of the conditioned reflex theory was a turning point in the history of natural sciences that transformed a human thought from a philosophy subject onto a subject of experimental physiological research. The formation of conditioned reflexes is the main physiological mechanism that defines the production of new attitudes of an organism to the surrounding environment during the life of the organism.

Several conditions are needed for the development of the conditioned reflex. Firstly, two stimuli are provided, namely an unconditioned stimulus (e.g., food, pain stimuli, and so forth) that induces an unconditioned reflex reaction and a conditioned stimulus (e.g., light, sound, appearance of the food, and the like) that signals about the unconditioned stimulus to come. Secondly, the conditioned and unconditioned stimuli are combined multiple times. In some case, the conditioned reflex may be formed upon a single combining of the conditioned stimulus and the unconditioned stimulus.

Thirdly, the conditioned stimulus must precede the action of the unconditioned stimulus. Fourthly, any stimulus of the external or internal environment can be used as a conditioned stimulus. Specifically, the stimulus needs to be indifferent, not causing a defensive reaction of a person, having no excessive strength, and may be able to attract attention of the person. Fifthly, the selected unconditioned stimulus needs to be strong, otherwise the temporary connection between the conditioned stimulus and the unconditioned stimulus may not be formed. Sixthly, the stimulation caused by the unconditioned stimulus needs to be stronger than the stimulation caused by the conditioned stimulus. Seventhly, extraneous stimuli need to be removed since they can cause the slowdown of formation of the conditioned reflex. Eighthly, the person who develops a conditioned reflex has to be healthy. Ninthly, when developing the conditioned reflex, the person needs to be highly motivated. For example, when developing a food reflex, the person must be hungry because in case the person is well-fed, the conditioned reflex cannot be formed.

It is easier to produce conditioned reflexes to effects that are ecologically close to the person. In view of this, conditioned reflexes include natural conditioned reflexes and artificial conditioned reflexes. Natural conditioned reflexes are produced in response to agents that naturally act along with a stimulus that causes an unconditioned reflex (for example, the appearance of food, the smell of the food, etc.). All other conditioned reflexes are artificial, i.e. are produced by agents that are not normally associated with the action of an unconditioned stimulus, for example, a food salivary reflex to bell ringing.

The theory of signaling systems. After a detailed analysis of the higher nervous activity of animals, Pavlov determined specific features of the higher nervous activity of a human, as a result of which Pavlov formulated an idea of two signaling systems. According to Pavlov, the first signaling system includes all temporary connections resulting from the co-occurrence of real stimuli with any activity of the organism. In this case, various visual, auditory, tactile and other stimuli are considered signals of the imminent formation of unconditioned stimuli.

The first signaling system is the system of an organism that provides the formation of a particular, direct understanding of the surrounding environment and formation of adaptive reactions through conditional connections. The signals of the first signal system are objects, phenomena and their individual properties (smell, color, shape, and so forth).

The second signaling system includes all speech temporary associations that are formed as a result of the co-occurrence of words with an action of direct real stimuli or with other words. The second signaling system is the system of the organism that provides the formation of a generalized understanding of the surrounding environment using the human language. The human language is a means of communication of people with each other, the main form of which is a written and oral speech. The speech is a form of communication of people with each other with the help of signals (words) that ensures human thinking. The speech can be internal, which is a necessary form of the thinking process, and external (written and oral), by which a person communicates his thoughts to other people.

Three levels of higher nervous activity of a person can be determined. The first level is the level of unconditioned reflexes and instincts, which are caused by relatively few stimuli. The anatomical basis of this activity is the spinal cord and most of the brain regions. The second level is the level of conditioned reflexes produced in the course of individual life of a person in response to various stimuli, which serve as signals about the possible occurrence of a particular reinforcement. The cerebral cortex is responsible for these reflexes. The third level is the level of the verbal signaling. The anatomical substrate of the level of the verbal signaling is the associative zones of the cerebral cortex (frontal, parietal), as well as higher sensory and motor centers of the cerebral cortex. For a person, a word is the same real factor as all other stimuli affecting the body, the word has the property of replacing real unconditioned and conditioned stimuli by functioning as a “signal of signals”. In addition, words are symbols of real stimuli, which, in turn, are signals of the imminent occurrence of reinforcement of the stimuli. This feature of the words made them signs, i.e., symbols of a huge number of stimuli of the first signal system. In an adult, the second signaling system is responsible for speech and all derivatives of speech, including character drawings, sign language, and facial expressions.

As used herein, an attitude is a subconscious state of dynamic predisposition to an action. The attitude may be divided into three levels: behavioral level, emotional level, and cognitive level.

An attitude pattern is a set of attitudes diagnosed in the behavior of a person.

A natural environment of psychological correction relates to performing behavior modification in real life conditions, in which the unconditioned stimulus, for example, is the food reflex, and the process of conditioning the new stimulus is performed during the food intake.

An artificial environment of psychological correction relates to performing behavior modification in any environment. In this case, the unconditional stimulus is sensory stimuli and the conditioned stimulus is formed by the visual and auditory speech channel.

A cognitive work with a patient includes providing daily counseling to the patient by a psychologist during the period of behavior modification in order to motivate the patient to recover and adhere to the steps of the behavior modification method. The counseling may include providing periodical consultations (e.g., daily, weekly, monthly) by the psychologist to the patient by communicating with the patient via a computer, phone, messengers, such as Skype™, video chat programs, and so forth.

The systems and methods of the present disclosure may be used for providing remote physiological and psychotherapeutic aid to patients. Furthermore, the systems and methods may be used in teletherapy and, in particular, in telepsychology. Telepsychology includes providing physiological and psychotherapeutic aid remotely via communication means, such as a computer, a phone, and so forth. The systems and methods are aimed at improving the quality of life of persons by modifying the behavior treated as undesirable by the persons.

The systems and methods of the present disclosure can be used in the traditional practice of cognitive behavioral therapy. When applied in traditional cognitive behavioral therapy, the systems and methods may be embedded into the behavior modification process as one of the methods of behavior modification.

The effectiveness of the systems and methods when applied remotely or when applied in traditional cognitive behavioral therapy is the same. The ability to work with a patient remotely allows the systems and methods to be used in cases when the use of traditional methods of psychotherapy is problematic.

The systems and methods are advantageous when applied in cases of burnout of a person when working with problematic clients. Other applications include providing aid to sensitive clients who suffer when talking on sensitive topics. Furthermore, the systems and methods may be advantageously used for providing aid to clients who wish to receive psychological aid in home conditions. In addition, the systems and methods may be used for providing services to people living in remote places and having no access to medical centers.

Additional advantages of the systems and methods of the present disclosure include reducing the complexity of providing psychological aid to a person by a therapist and facilitating the selection of conditional stimuli for the person by the therapist.

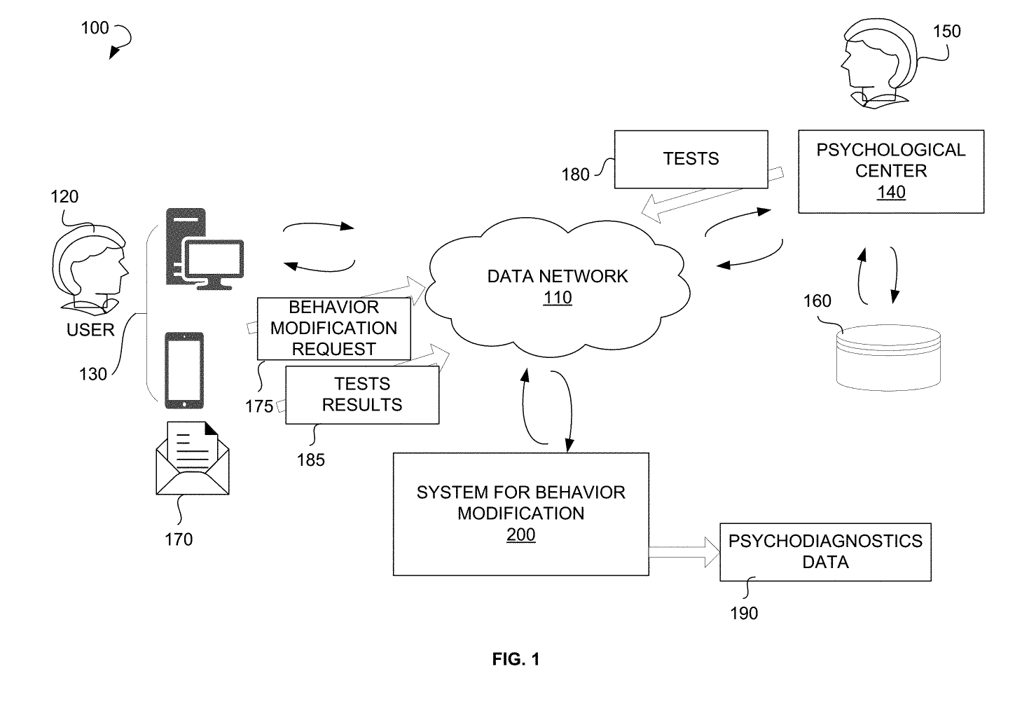

Referring now to the drawings, FIG. 1 illustrates an environment 100 within which methods and systems for behavior modification can be implemented. The environment 100 may include a data network 110, a patient 120, a user device 130, a psychological center 140, a psychologist 150 associated with the psychological center 140, a database 160 associated with the psychological center 140, and a system 200 for behavior modification also referred to as a system 200. The user device 130 may include a personal computer (PC), a laptop, a smartphone, a tablet PC, a personal wearable device, and so forth. The psychological center 140 may be associated with a website (not shown) of the psychological center 140.

The data network 110 may include the Internet, a computing cloud, and any other network capable of communicating data between devices. Suitable networks may include or interface with any one or more of, for instance, a local intranet, a Personal Area Network, a Local Area Network, a Wide Area Network, a Metropolitan Area Network, a virtual private network, a storage area network, a frame relay connection, an Advanced Intelligent Network connection, a synchronous optical network connection, a digital T1, T3, E1 or E3 line, Digital Data Service connection, Digital Subscriber Line connection, an Ethernet connection, an Integrated Services Digital Network line, a dial-up port such as a V.90, V.34 or V.34bis analog modem connection, a cable modem, an Asynchronous Transfer Mode connection, or a Fiber Distributed Data Interface or Copper Distributed Data Interface connection. Furthermore, communications may also include links to any of a variety of wireless networks, including Wireless Application Protocol, General Packet Radio Service, Global System for Mobile Communication, Code Division Multiple Access or Time Division Multiple Access, cellular phone networks, Global Positioning System, cellular digital packet data, Research in Motion, Limited duplex paging network, Bluetooth radio, or an IEEE 802.11-based radio frequency network. The data network can further include or interface with any one or more of Recommended Standard 232 (RS-232) serial connection, an IEEE-1394 (FireWire) connection, a Fiber Channel connection, an IrDA (infrared) port, a Small Computer Systems Interface connection, a Universal Serial Bus connection or other wired or wireless, digital or analog interface or connection, mesh or Digi® networking. The data network may include a network of data processing nodes, also referred to as network nodes, that are interconnected for the purpose of data communication.

The patient 120 may register with the website of the psychological center 140 using a communication means (for example, the user device 130 or any other communication means, such as by mail 170). The psychological center 140 may provide psychological aid to patients remotely, i.e., without a direct contact of patients with psychologists. Upon registering, the patient 130 may send a behavior modification request 175 to the website of the psychological center 140.

After that, the patient 120 may be remotely exposed to psychodiagnostics using tests. Specifically, the psychological center 140 may provide tests 180 to the patient 120. The patient 120 may answer the test questions by himself and send test results 185 to the psychological center 140. After receiving the test results 185, the psychologist 150 of the psychological center 140 may consider the test results 185 as psychodiagnostics data 190 of the patient 120. The psychologist 150 may use the psychodiagnostics data 190 to develop a behavior modification training individually for the patient 120.

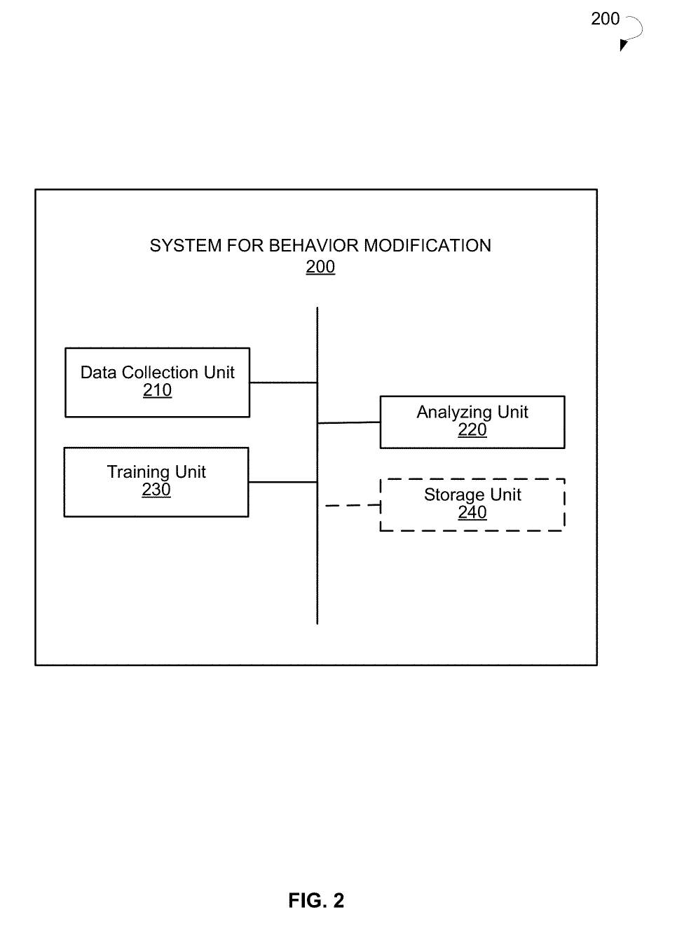

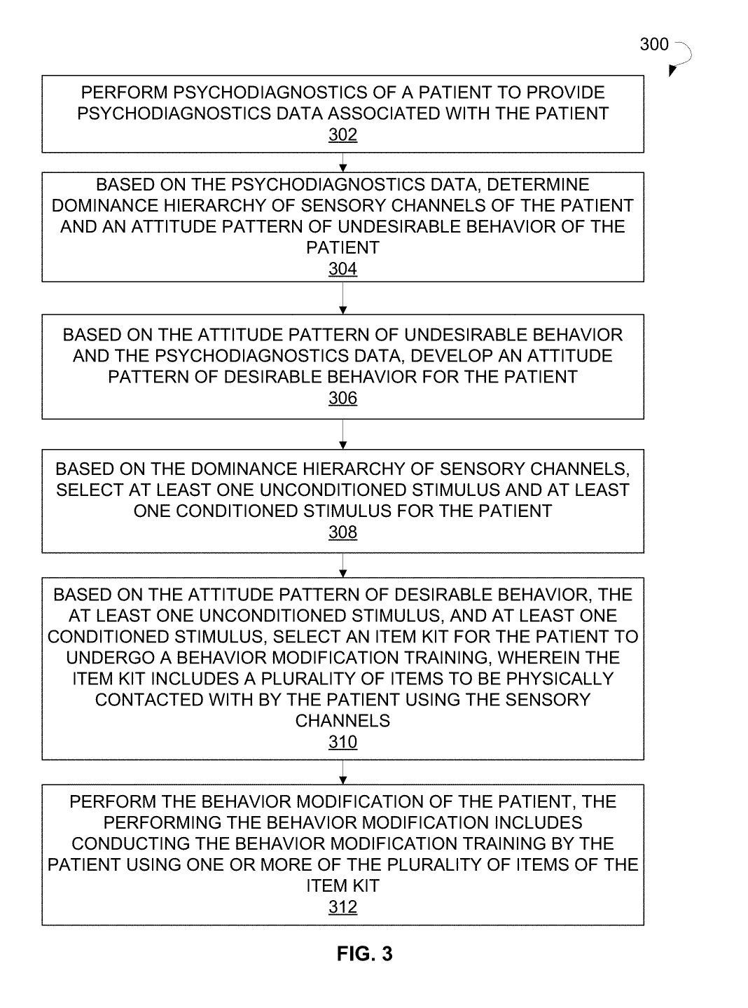

FIG. 2 is a block diagram showing various modules of a behavior modification system 200, in accordance with certain embodiments. Specifically, the system 200 may include a data collection unit 210, an analyzing unit 220, a training unit 230, and optionally a storage unit 240. In an example embodiment, each of the data collection unit 210, the analyzing unit 220, and the training unit 230 may include a programmable processor, such as a microcontroller, a central processing unit, and so forth. In example embodiments, each of the data collection unit 210, the analyzing unit 220, and the training unit 230 may include an application-specific integrated circuit or programmable logic array designed to implement the functions performed by the system 200. Operations performed by each of units of the system 200 are described below with reference to FIG. 3. In an example embodiment, the data collection unit 210, the analyzing unit 220, the training unit 230, and the storage unit 240 may perform the operations in associated with a website of a psychological center.

FIG. 3 is a flow chart illustrating a behavior modification method 300, in accordance with certain embodiments. The method 300 may optionally include registering a patient with a website of a psychological center. The psychological center may provide a behavior modification aid to patients. Furthermore, the patient and the psychological center may enter into an agreement on providing psychological aid by the psychological center to the patient. Upon registering, the patient may send a request for the behavior modification. The patient may send the request for the behavior modification via a communication means, for example, by accessing the website using a computer of the patient. A data collection unit may receive the request for the behavior modification provided by the patient using the website.

The method 300 may continue with performing psychodiagnostics of the patient to provide psychodiagnostics data associated with the patient at operation 302. Specifically, based on the request, the data collection unit may send, via the communication means, one or more tests to perform psychodiagnostics to the patient. hi an example embodiment, the one or more tests may be provided to the patient by e-mail or by mail. In another example embodiment, the patient may access the tests on the website and answer questions of the tests directly on the website. The tests may include questions required for the psychologist to obtain all psychodiagnostics data needed for performing the behavior modification of the patient. The patient may receive the tests, answer questions of the tests by himself, and send the test results back to the psychologist.

The method 300 may continue with receiving, via the communication means, results of the one or more tests from the patient. The results of the one or more tests may be considered the psychodiagnostics data of the patient. In an example embodiment, the results of the one or more tests may be additionally analyzed by an analyzing unit. In a further example embodiment, the results of the one or more tests may be additionally analyzed by the psychologist.

The method 300 may further include determining dominance hierarchy of seven sensory channels of the patient and determining an attitude pattern of undesirable behavior of the patient at operation 304. The dominance hierarchy of sensory channels and the attitude pattern of undesirable behavior may be determined based on the psychodiagnostics data of the patient. The dominance hierarchy of sensory channels shows which sensory channels of the patient are dominant. For example, based on the psychodiagnostics data, it may be determined that the auditory sensory channel is a primary dominant sensory channel and the visual sensory channel is a secondary dominant sensory channel of the patient. The term “dominant sensory channel” shall mean a sensory channel through which the patient is stimulated in response to stimuli of the environment stronger and faster than through other sensory channels. Other sensory channels of the patient may be non-dominant. The attitude pattern of undesirable behavior of the patient may include overeating, gambling, alcohol abuse, drug abuse, smoking, and so forth. The attitudes may be classified into emotional, behavioral, and cognitive.

The method 300 may continue with developing an attitude pattern of desirable behavior for the patient at operation 306. The attitude pattern of desirable behavior may be selected or developed by the psychologist or the analyzing unit based on the attitude pattern of undesirable behavior and the psychodiagnostics data. The attitude pattern of desirable behavior may include developing healthy eating habits, non-aggressive behavior, withdrawal from alcohol or drugs, withdrawal from smoking, regular physical trainings, and so forth.

At operation 308, at least one unconditioned stimulus and at least one conditioned stimulus for the patient may be selected based on the dominance hierarchy of sensory channels. In an example embodiment, the at least one unconditioned stimulus may include one or more of the following: a food stimulus, a pain stimulus, a smell stimulus, a light stimulus, a sensor stimulus, and so forth. The at least one conditioned stimulus may include one or more of the following: an auditory stimulus, a visual stimulus, a tactile stimulus, an olfactory stimulus, a kinesthetic stimulus, and a gustatory stimulus.

The method 300 may further include selecting an item kit for the patient to undergo a behavior modification training at operation 310. The item kit may be selected based on the attitude pattern of desirable behavior, the at least one unconditioned stimulus, and at least one conditioned stimulus. The item kit may include a plurality of items to be physically contacted with by the patient using the sensory channels. The item kit may be selected as follows. Firstly, one or more items to act as the at least one unconditioned stimulus and at least one conditioned stimulus for the patient may be determined through the sensory channels of the patient. Secondly, a number of the items required to stimulate the patient through the sensory channels may be determined. In an example embodiment, the plurality of items of the item kit may include one or more of the following: a text for audio listening, an audio file, at least one food additive, at least one aromatic essential oil, at least one mineral, at least one image, and so forth. In an example embodiment, the image may include a suggestive formula to develop the attitude pattern of desirable behavior for the patient.

The selected item kit may further include tools, or accessories, for using one or more of the plurality of items, such as an aroma lamp for the aromatic essential oil and a voice recorder for recording the text by the patient.

Additionally, the method 300 may include selecting an environment for conducting the behavior modification training. The environment may be selected from a natural environment and an artificial environment. The selection may be made based on the psychodiagnostics data, the attitude pattern of undesirable behavior, and the attitude pattern of desirable behavior. The classical conditioning can be performed both in the natural environment, where there is a natural unconditioned stimulus, and in the artificial environment, where the stimuli selected by the psychologist replace the behavioral scenarios.

The method 300 may include developing a program of the behavior modification training to condition a neutral stimulus of the patient, which further becomes a conditioned stimulus upon creation of the association between an unconditioned stimulus and the neutral stimulus. The program may include a list of actions to be performed by the patient using the item kit, instructions on use of the item kit, a schedule of performing the behavior modification training, and a duration of the behavior modification training. The program of the behavior modification training and the item kit may be provided to the patient. For example, the program of the behavior modification training and the item kit may be sent to the patient by mail.

The method 300 may continue with performing the behavior modification of the patient at operation 312. The behavior modification may be performed by conducting the behavior modification training by the patient using one or more of the plurality of items of the item kit. In an example embodiment, the psychologist and the patient may establish an online contact between them, for example, using the website, a phone, any messengers, or video chat programs. The psychologist may regularly consult the patient with regard to undergoing the behavior modification training by the patient and monitor the behavior modification progress of the patient.

The behavior modification training may be performed by the patient for example, for six weeks, though a sustained remission can be observed after two weeks or, in some cases, even the next day. The patient may perform the behavior modification training two or three times a day. An example duration of the behavior modification training may be up to 20 minutes. The patient performs the behavior modification training by himself. The conditioned reflex of the highest level developed in the process of classical conditioning is associated with the attitude pattern. Each attitude is conditioned independently. The attitude pattern affects, on an unconscious level, the new modified behavior. Upon developing the conditioned reflex of the highest level in the patient, the conditioned reflex is felt by the patient as a desire to act according to the modified behavior.

To avoid the effect of “slowdown”, in the case of application of several stimuli, unconditioned reflexes and conditioned reflexes of the first level can be changed sequentially during the behavior modification training. The attitude pattern of the conditioned reflex of the highest level remains unchanged during the whole course of behavior modification.

In psychology, the attitude has three levels. Specifically, behavioral, cognitive, and emotional levels of the attitude are determined. The attitudes of the attitude pattern of the highest level conditional reflex are selected by the psychologist based on psychodiagnostics data of the patient. The attitude pattern of the conditioned reflex needs to be sufficient for strong control of the modified behavior.

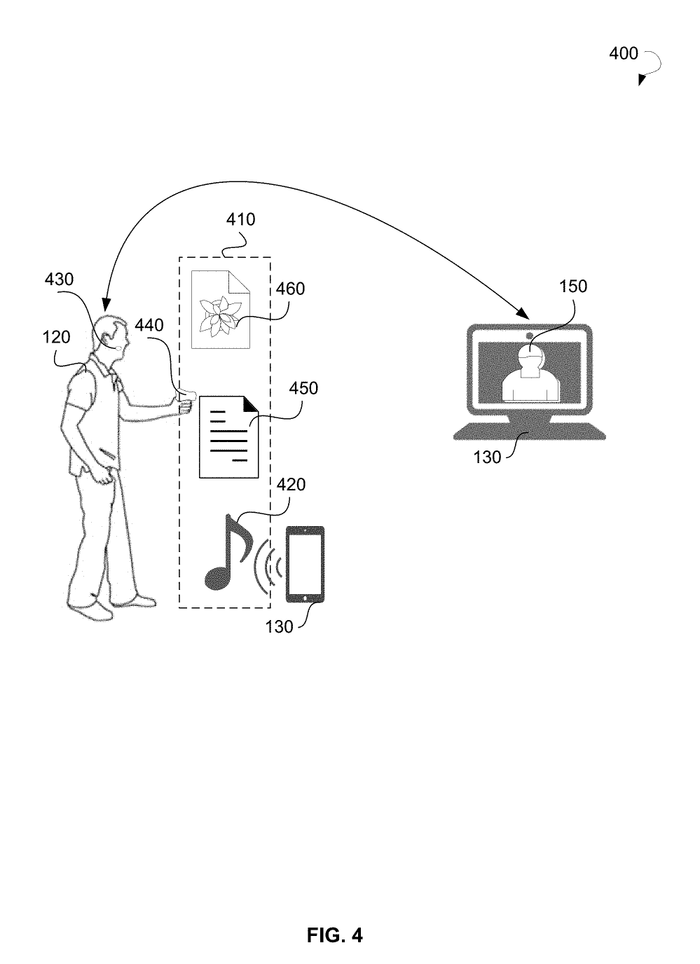

FIG. 4 is a schematic diagram 400 illustrating the behavior modification of a patient using an item kit, according to an example embodiment. The patient 120 may receive an item kit 410 from a psychological center. The item kit 410 may include a plurality of items and instructions on use of the item kit. Each of the items may be selected based on psychodiagnostics data of the patient obtained based on psychodiagnostics of the patient. Each of the items may be selected to act as at least one unconditioned stimulus or at least one conditioned stimulus for the patient using sensory channels of the patient. The items may be selected from a group comprising: a text for audio listening, at least one food additive, at least one aromatic essential oil, at least one mineral, and one or more images with a suggestive formula to develop an attitude pattern of desirable behavior of a patient. The text 450 may be drafted individually for the patient 120 by the psychologist 150 based on the psychodiagnostics data of the patient 120 and may be directed to developing the attitude of desirable behavior. Upon receipt of the item kit, the patient may review the instructions concerning the use and perform necessary actions. In an example embodiment, the patient 120 may use a voice recorder or an audio player to record the text 450 for audio listening as an audio file 420 using a voice of the patient 120. The text 450 recorded as the audio file 420 may stimulate the patient 120 through an auditory sensory channel of the sensory system.

The food additive 430 may have a strong taste. The food additive 430 may be a food additive pleasant for the patient 120, for example, a mint lollipop, which may cause a long pleasant taste effect. The food additive 430 may be selected based on the psychodiagnostics of the patient 120 from variants provided in tests. The food additive 120 may stimulate the patient 120 through a gustatory sensory channel of the sensory system.

The item kit 410 may further include a device for using the aromatic essential oils, such as an aroma lamp. Particular aromatic essential oils may be selected in the course of remote psychodiagnostics and may be later used by the patient 120 or the psychologist to create a persistent aroma in the room while undergoing the behavior modification training by the patient 120. The essential oils may stimulate the patient 120 through an olfactory sensory channel of the sensory system of the patient.

The mineral 400 may include minerals (e.g., stones) of various shapes. The minerals may provide various tactile sensations. The mineral 440 may be selected by the patient 120 or the psychologist from variants provided in tests during the psychodiagnostics. The mineral 440 may be held by the patient 120 in his hand while listening to the text 450. The mineral 400 may stimulate the patient 120 through a tactile sensory channel of the sensory system.

The one or more images 460 may have a suggestive formula. The suggestive formula may be selected to develop a new attitude of the patient. A first image may be an image made according to a design determined based on the psychodiagnostics. The first image may include one or more linguistic phrases of the suggestive formula to form the new attitude. The patient 120 may choose a particular image, as well as color and text font, which are the most pleasant to him, by himself during the psychodiagnostics. The image may be selected to stimulate (e.g., evoke pleasant impressions) the patient 120 through a visual sensory channel of the sensory system of the patient.

A second image may include a suggestive formula of kinesthetic sensations. The second image may be made according to a design determined based on the psychodiagnostics. The second image may include one or more linguistic phrases targeting kinesthetic sensations, the character of which may be also determined during the testing. The patient 120 may choose a particular image, as well as color and text font, which are the most pleasant to him, by himself during the psychodiagnostics. The second image may stimulate (e.g., evoke pleasant impressions) the patient 120 through the kinesthetic sensory channel of information perception.

A third image may have a suggestive formula of reasoned persuasions. The third image may be made according to a design determined based on the psychodiagnostics. The patient 120 selects the design and particular image from variants provided in tests by himself during the psychodiagnostics. The third image may be based on linguistic phrases, or reasoned persuasions. The reasoned persuasions may be also formed based on tests and may focus the attention of the patient during the behavior modification training on pleasant reasoned persuasions. The third image may stimulate (e.g., evoke pleasant impressions) the patient 120 through a reasoning sensory channel of information perception.

Sensor reactions of the patient 120 to the food additive 430 and the mineral 440 may be selected as unconditioned stimuli. The sensor reactions of the patient 120 to listening to the audio file 420 and viewing the image 460 may be used as conditioned stimuli. Both the audio file 420 and the image 460 may be associated with the attitude of desirable behavior. For example, an attitude of undesirable behavior of the patient 120 may include overeating. The attitude of desirable behavior selected by the psychologist to be formed in the patient may include developing healthy eating habits. In view of this, the audio file 420 may include the text stimulating the patient 120 to develop healthy habits, e.g., “I eat regularly every day.” The image 460 may include an image of fresh vegetables and fruits and an image of a healthy person.

The patient 120 may be exposed to simultaneous stimulation by the unconditioned stimuli and conditioned stimuli. Upon repeating training sessions for a predetermined period of time, e.g., for six weeks daily, the attitude of the desirable behavior may be formed based on developed conditioned stimuli. For example, the person 120 may develop healthy eating habits.

The psychologist 150 may be in online contact with the patient 120 during or after every session of the behavior modification training and may provide further recommendations and explanations to the patient 120.

Behavior modification in an artificial environment. The behavior modification training may be performed by the patient two or three times a day. The patient may be located in a room where no one distracts the attention of the patient and may apply the item kit provided to the patient. The duration of the behavior modification training may be 20 minutes. Unconditional stimuli may be selected by a psychologist as sensory stimuli of the patient in response to contacting with items of the item kit, and conditional stimuli may be in form of auditory and visual information. The psychologist may communicate daily with the patient online, motivate the client to work with the item kit, support high emotional tone of the patient, and monitor the correct control of attention by the patient in the course of the behavior modification method to make the patient to focus on the items.

Behavior modification in a natural environment. The behavior modification training may be performed by the patient once or twice a day. The patient may create the natural environment of an unconditioned stimulus (for example, the process of eating). A conditioned stimulus may be present in form of auditory and visual information. The psychologist may communicate daily with the patient online, motivate the patient to work with the item kit, support high emotional tone of the patient, and monitor the correct control of attention by the patient in the course of the behavior modification method to make the patient to focus on the items.

### Example of Conducting a Behavior Modification Method in an Artificial Environment

A 45-year-old man having the problem of being overweight applied for help. The behavior modification method is directed to modifying the behavior related to overeating. The patient registered with the website and signed a service agreement. Upon registering, psychodiagnostics data of the patient were collected by providing tests to the patient and answering 12 test questions by the patient. Based on the test results, the psychologist determined that the behavior modification method needs to be conducted in an artificial environment. To create the unconditioned reflex, mint aroma (mint essential oil) and mint taste (mint candies) were chosen, a pleasant design table with a visual suggestive formula was selected, and a tactile stimulus in form of green glass beads was selected. The goal of behavior modification method was to condition the pattern of attitudes aimed at reducing weight. The attitude pattern is formed as a conditioned stimulus with the help of audible and visual signals. When performing psychodiagnostics, it was determined that the following set of attitudes is responsible for overweight: extra eating, eating fast, eating before bedtime, eating high-calorie food, enjoying overeating, having a non-mobile lifestyle, and being comfortable when having the non-mobile lifestyle. In view of this, the attitude pattern at which the person may lose weight was developed for the patient. In particular, the attitude of desirable behavior included: eating less, eating slowly, observing the nutrition discipline, eating low-calorie food, enjoying a diet, having a mobile lifestyle, and enjoying playing sports. An item kit required for the behavior modification was sent to the patient as a package. The psychologist established an online psychotherapeutic contact with the patient and provided preliminary instructions on the behavior modification training.

The behavior modification training was conducted according to the classical conditioning method. The patient by himself performed the behavior modification training based on the received instructions and received further instructions from the psychologist several times online. Sensory responses to items of the item kit were used as unconditioned reflexes, and audio information and video information directed to the pattern of new attitudes defining the new modified behavior were used as a conditioned reflex.

In the course of behavior modification training, the patient fell into a state of apathy three times and demonstrated avoidance reactions and unwillingness to carry out procedures in due course. In all cases, the psychologist successfully performed the cognitive work with the patient, supported the motivation of the patient to motivate the patient to continue the behavior modification training. On the tenth day, steady remission occurred and the amount of food consumed by the patient reduced to the predicted amount. The type of the food selected by the patient for eating turned to dietary. On the twelfth day, the physical activity of the patient increased by 45% and the weight of the patient began to reduce. The weight returned to normal on the 36th day of the behavior modification training. The weight of the patient decreased by 7 kg. The patient left a positive review on the website of the psychological center.

### Example of Conducting a Behavior Modification Method in a Natural Environment

A 29-year-old patient applied for help. The patient had the undesirable behavior including gambling, a complicated form of dependency associated with loss of social status, breaking family relationships, dismissal from work, and depression. The patient registered with the website and signed a service agreement. Upon registering, psychodiagnostics data of the patient were collected by providing tests to the patient and answering 16 test questions by the patient. Based on the test results, the psychologist determined that the behavior modification method needs to be conducted in a natural environment. The classical conditioning method took place twice a day, namely in the morning during breakfast and in the evening during lunch. The aim of the behavior modification method was to condition the pattern of attitudes aimed at developing the behavior without gambling dependency, solving the problems in personal relationships in the family and in society. The conditional stimulus being the attitude pattern of the new behavior was formed using an auditory signal and visual signal, namely an audio recording and a visual table with a suggestive formula for modified behavior. In the course of psychodiagnostics, it was determined that the reasons for gambling addiction include the following set of attitudes: lack of attention from friends, problematic relations with the wife of the patient, conflicts at work, lack of funds, and tendency to risk. An attitude pattern of modified behavior was developed at which the passion for gambling was lost and social relations returned. The attitude pattern of modified behavior included creating relationships with friends, finding a job with desired salary, returning to the family, developing a negative attitude to risk, and playing sports. An item kit necessary for the behavior modification was sent to the patient in a package. The psychologist established online psychotherapeutic contact with the patient and provided preliminary instructions on the behavior modification training.

The behavior modification training was conducted according to the classical conditioning method. The patient by himself performed the behavior modification training based on the received instructions and received further instructions from the psychologist several times online. Sensory responses to food were used as unconditioned reflexes, and audio information and video information directed to the pattern of new attitudes defining the new modified behavior were used as a conditioned reflex.

The conduction of the behavior modification training had some difficulties. The patient violated the instructions for the first six days. The psychologist had to perform cognitive work (e.g., providing additional explanations and motivating) with the patient for 30-40 minutes daily. The patient often fell into an apathy state and 5 times had avoidance reactions and unwillingness to work as required by the instructions. In all cases, the cognitive work of the psychologist with the patient was successful, the psychologist supported the motivation of the patient, and the behavior modification course continued. On day 16, signs of remission appeared and interest of the patient in gambling began to reduce. On day 22, the patient began to search for a job. On day 25, a relationship of the patient with a friend was restored. On day 31, the patient had the first contact with his wife. On day 33, the patient found a job. On day 36, the patient started running. On day 39, the patient returned to his family. On day 41, the patient had a steady indifferent attitude to the gambling. The behavior modification process was completed. The patient left a positive review on the website of the psychological center.

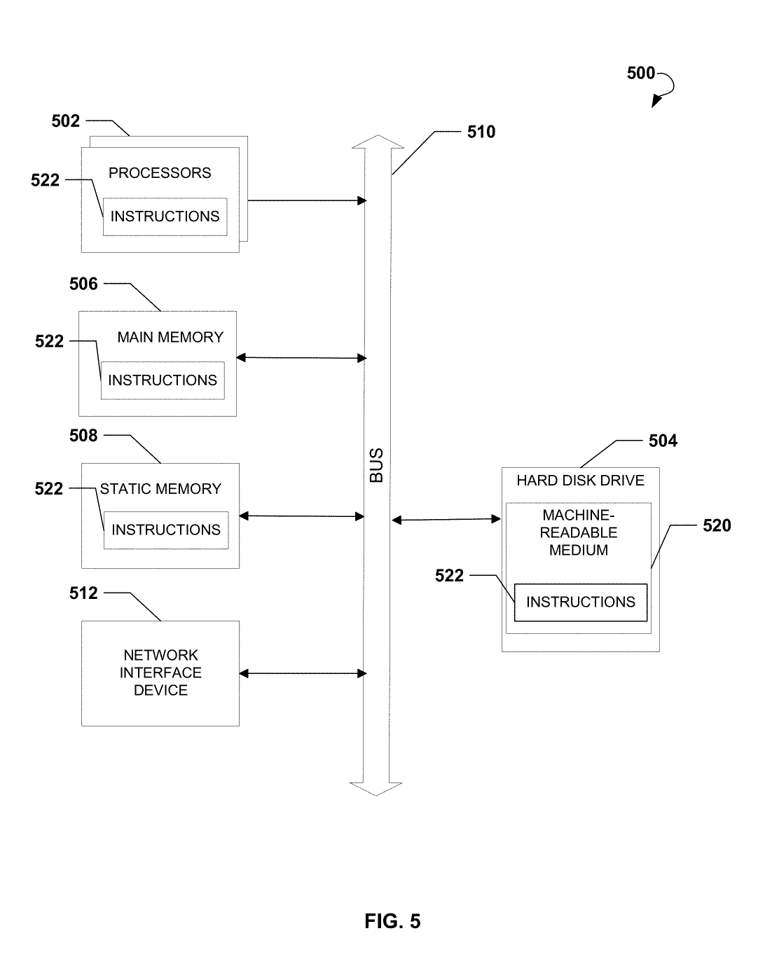

FIG. 5 shows a diagrammatic representation of a computing device for a machine in the exemplary electronic form of a computer system 500, within which a set of instructions for causing the machine to perform any one or more of the methodologies discussed herein can be executed. In various exemplary embodiments, the machine operates as a standalone device or can be connected (e.g., networked) to other machines. In a networked deployment, the machine can operate in the capacity of a server or a client machine in a server-client network environment, or as a peer machine in a peer-to-peer (or distributed) network environment. The machine can be a PC, a tablet PC, a set-top box, a cellular telephone, a digital camera, a portable music player (e.g., a portable hard drive audio device, such as a Moving Picture Experts Group Audio Layer 3 (MP3) player), a web appliance, a network router, a switch, a bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The computer system 500 may include a processor or multiple processors 502, a hard disk drive 504, a main memory 506 and a static memory 508, which communicate with each other via a bus 510. The computer system 500 may also include a network interface device 512. The hard disk drive 504 may include a computer-readable medium 520, which stores one or more sets of instructions 522 embodying or utilized by any one or more of the methodologies or functions described herein. The instructions 522 can also reside, completely or at least partially, within the main memory 506 and/or within the processors 502 during execution thereof by the computer system 500. The main memory 506 and the processors 502 also constitute machine-readable media.

While the computer-readable medium 520 is shown in an exemplary embodiment to be a single medium, the term “computer-readable medium” should be taken to include a single medium or multiple media (e.g., a centralized or distributed database, and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media. Such media can also include, without limitation, hard disks, floppy disks, NAND or NOR flash memory, digital video disks, Random Access Memory, Read-Only Memory, and the like.

The example embodiments described herein may be implemented in an operating environment comprising software installed on a computer, in hardware, or in a combination of software and hardware.

In some embodiments, the computer system 500 may be implemented as a cloud-based computing environment, such as a virtual machine operating within a computing cloud. In other embodiments, the computer system 500 may itself include a cloud-based computing environment, where the functionalities of the computer system 500 are executed in a distributed fashion. Thus, the computer system 500, when configured as a computing cloud, may include pluralities of computing devices in various forms, as will be described in greater detail below.

In general, a cloud-based computing environment is a resource that typically combines the computational power of a large grouping of processors (such as within web servers) and/or that combines the storage capacity of a large grouping of computer memories or storage devices. Systems that provide cloud-based resources may be utilized exclusively by their owners or such systems may be accessible to outside users who deploy applications within the computing infrastructure to obtain the benefit of large computational or storage resources.

The cloud may be formed, for example, by a network of web servers that comprise a plurality of computing devices, such as the computer system 500, with each server (or at least a plurality thereof) providing processor and/or storage resources. These servers may manage workloads provided by multiple users (e.g., cloud resource customers or other users). Typically, each user places workload demands upon the cloud that vary in real-time, sometimes dramatically. The nature and extent of these variations typically depends on the type of business associated with the user.

Thus, methods and item kits for behavior modification are described. Although embodiments have been described with reference to specific exemplary embodiments, it will be evident that various modifications and changes can be made to these exemplary embodiments without departing from the broader spirit and scope of the present application. Accordingly, the specification and drawings are to be regarded in an illustrative rather than a restrictive sense.

What is claimed is:

1. A method for behavior modification, the method comprising:
performing psychodiagnostics of a patient to provide psychodiagnostics data associated with the patient;
based on the psychodiagnostics data, determining dominance hierarchy of sensory channels of the patient and an attitude pattern of undesirable behavior of the patient;
based on the attitude pattern of undesirable behavior and the psychodiagnostics data, developing an attitude pattern of desirable behavior for the patient;
based on the dominance hierarchy of sensory channels, selecting at least one unconditioned stimulus and at least one conditioned stimulus for the patient;
based on the attitude pattern of desirable behavior, the at least one unconditioned stimulus, and at least one conditioned stimulus, selecting an item kit for the patient to undergo a behavior modification training, wherein the item kit includes a plurality of items to be physically contacted with by the patient using the sensory channels;
performing the behavior modification of the patient, the performing the behavior modification includes conducting the behavior modification training by the patient using one or more of the plurality of items of the item kit.

2. The method of claim 1, further comprising:
receiving, via a communication means, a request for the behavior modification from the patient;
based on the request, sending, via the communication means, one or more tests to perform the psychodiagnostics to the patient.

3. The method of claim 2, wherein the request for the behavior modification is received from the patient using a website associated with a psychological center, the psychological center providing a behavior modification aid.

4. The method of claim 2, further comprising receiving, via the communication means, results of the one or more tests from the patient, wherein the psychodiagnostics data include the results of the one or more tests.

5. The method of claim 1, wherein the at least one unconditioned stimulus includes one or more of the following: a food stimulus, a pain stimulus, a smell stimulus, a light stimulus, and a sensor stimulus.

6. The method of claim 1, wherein the at least one conditioned stimulus includes one or more of the following: an auditory stimulus, a visual stimulus, a tactile stimulus, an olfactory stimulus, a kinesthetic stimulus, and a gustatory stimulus.

7. The method of claim 1, further comprising:
developing a program of the behavior modification training, the program including a list of actions to be performed by the patient using the item kit, instructions on use of the item kit, a schedule of performing the behavior modification training, and a duration of the behavior modification training.

8. The method of claim 1, further comprising providing the program of the behavior modification training and the item kit to the patient.

9. The method of claim 1, further comprising:
based on the psychodiagnostics data, the attitude pattern of undesirable behavior, and the attitude pattern of desirable behavior, selecting an environment for conducting the behavior modification training, the environment including a natural environment and an artificial environment.

10. The method of claim 1, wherein the selecting the item kit includes:
determining one or more items to act as the at least one unconditioned stimulus or at least one conditioned stimulus for the patient through the sensory channels; and
determining a number of the one or more items required to stimulate the patient through the sensory channels.

11. The method of claim 1, wherein the plurality of items of the item kit includes one or more of the following: a text for audio listening, an audio file, at least one food additive, at least one aromatic essential oil, at least one mineral, and at least one image.

12. The method of claim 10, wherein the at least one image includes a suggestive formula to develop the attitude pattern of desirable behavior for the patient.

13. A system for behavior modification, the system comprising:
a data collection unit configured to perform psychodiagnostics of a patient to provide psychodiagnostics data associated with the patient;
an analyzing unit configured to:
based on the psychodiagnostics data, determine dominance hierarchy of sensory channels of the patient and an attitude pattern of undesirable behavior of the patient;
based on the attitude pattern of undesirable behavior and the psychodiagnostics data, develop an attitude pattern of desirable behavior for the patient;
based on the dominance hierarchy of sensory channels, select at least one unconditioned stimulus and at least one conditioned stimulus for the patient; and
based on the attitude pattern of desirable behavior, the at least one unconditioned stimulus, and at least one conditioned stimulus, select an item kit for the patient to undergo a behavior modification training, wherein the item kit includes a plurality of items to be physically contacted with by the patient using the sensory channels; and
a training unit configured to perform the behavior modification of the patient, the performing the behavior modification includes conducting the behavior modification training by the patient using one or more of the plurality of items of the item kit.

14. The system of claim 13, wherein the data collection unit is further configured to:
receive, via a communication means, a request for the behavior modification from the patient;
based on the request, send, via the communication means, one or more tests to perform the psychodiagnostics to the patient.

15. The system of claim 14, wherein the data collection unit is further configured to receive, via the communication means, results of the one or more tests from the patient, wherein the psychodiagnostics data include the results of the one or more tests.

16. The system of claim 13, wherein the analyzing unit is further configured to develop a program of the behavior modification training, the program including a list of actions to be performed by the patient using the item kit, instructions on use of the item kit, a schedule of performing the behavior modification training, and a duration of the behavior modification training.

17. The system of claim 13, wherein the analyzing unit is further configured to, based on the psychodiagnostics data, the attitude pattern of undesirable behavior, and the attitude pattern of desirable behavior for the patient, select an environment for conducting the behavior modification training, the environment including a natural environment and an artificial environment.

18. The system of claim 13, wherein the selecting the item kit includes:
determining one or more items to act as the at least one unconditioned stimulus or and at least one conditioned stimulus for the patient using the sensory channels; and
determining a number of the one or more items required to stimulate the patient through the sensory channels.

19. The system of claim 13, wherein the plurality of items of the item kit includes one or more of the following: a text for audio listening, an audio file, at least one food additive, at least one aromatic essential oil, at least one mineral, and at least one image.

20. An item kit for behavior modification, the item kit comprising:
a plurality of items, the plurality of items including one or more of:
a text for audio listening;
at least one food additive;
at least one aromatic essential oil;
at least one mineral; and
one or more images with a suggestive formula to develop an attitude pattern of desirable behavior of a patient; and
instructions on use of the item kit;
wherein each of the plurality of items is selected based on psychodiagnostics data of the patient, the psychodiagnostics data being obtained based on psychodiagnostics of the patient;
wherein each of the plurality of items is selected to act as at least one unconditioned stimulus or at least one conditioned stimulus for the patient using sensory channels of the patient.
